(19) United States
(12) Patent Application Publication
(10) Pub. No.: US 2026/0294984 A1
(43) Pub. Date: Oct. 1, 2026

(54) TRADITIONAL CHINESE MEDICINE COMPOSITION FOR TREATING POST-CORONARY HEART DISEASE DEPRESSION, PREPARATION METHOD AND APPLICATION THEREOF

(71) Applicant: Hangzhou Hongyu Pharmaceutical Technology Co., Ltd., Zhejiang (CN)

(72) Inventors: Haitong Wan, Zhejiang (CN); Yu He, Zhejiang (CN); Huifen Zhou, Zhejiang (CN); Jiehong Yang, Zhejiang (CN); Haofang Wan, Zhejiang (CN); Ningji Fang, Zhejiang (CN); Liquan Chen, Zhejiang (CN); Xinyu Zhao, Zhejiang (CN)

(73) Assignee: Hangzhou Hongyu Pharmaceutical Technology Co., Ltd., Zhejiang (CN)

(21) Appl. No.: 19/203,144

(22) Filed: May 8, 2025

(30) Foreign Application Priority Data
Mar. 31, 2025 (CN) 202510389576.X

(51) Int. Cl. A61K 35/618; A61K 31/045; A61K 36/48; A61K 36/537; A61K 36/634; A61K 36/8968; A61P 25/24

(52) U.S. Cl. CPC A61K 35/618; A61K 31/045; A61K 36/48; A61K 36/537; A61K 36/634; A61K 36/8968; A61P 25/24; A61K 2236/331; A61K 2236/53

(57) ABSTRACT

The invention relates to a Chinese medicine composition, a preparation method and application, which comprises the following components by weight: Ostreae concha 1500-1600 parts, Salviae miltiorrhizae radix et rhizoma 700-800 parts, Forsythiae fructus 600-700 parts, Dalbergiae odoriferae lignum 400-500 parts, Ophiopogonis radix 600-700 parts, Sappan lignum 400-500 parts and Borneolum syntheticum 10-11 parts. The Chinese medicine composition of the invention is suitable for the treatment of depression after myocardial infarction, can improve heart function, restore cognitive impairment, reduce negative emotion, stabilize the condition and improve the quality of life.

### CROSS-REFERENCE TO RELATED APPLICATION

This application claims the priority benefit of China application serial no. 202510389576.X, filed on Mar. 31, 2025. The entirety of the above-mentioned patent application is hereby incorporated by reference herein and made a part of this specification.

### BACKGROUND

### Technical Field

The invention belongs to the technical field of traditional Chinese medicine, in particular to a traditional Chinese medicine composition for treating post-coronary heart disease depression and a preparation method and application thereof.

### Description of Related Art

Nowadays, more and more people suffer from two or more long-term diseases at the same time, which is called polycomorbidities or comorbidity. At present, the situation of middle-aged and elderly people suffering from two or more diseases at the same time is becoming more and more serious, especially cardiovascular and cerebrovascular diseases and psychiatric diseases are often co-diseases and co-diseases. At present, the clinical prevalence of Post myocardial infarction depression (PMID) is as high as 79.5%. It has the characteristics of comorbidity and comorbidity, which seriously endangers human physical and mental health, and has become a common clinical major disease. At least half of the European population aged 65 or over (more than 50 million people) suffer from comorbidities. In the UK, the proportion of people with comorbidity is expected to rise from 54% in 2015 to 68% in 2035. However, the scientific and medical circles often aim at the diagnosis and treatment or research of a single disease in a specialized way of thinking, ignoring the internal relationship between etiology, pathogenesis and diagnosis and treatment in the same disease state, resulting in incomplete treatment strategies and programs.

Cardiovascular diseases often have comorbidities and co-diseases, and the number of deaths accounts for about ⅓ of all deaths in the world. Coronary atherosclerotic heart disease (CHD) Coronary heart disease, referred to as coronary heart disease, refers to coronary artery atherosclerosis caused by lumen stenosis or occlusion, resulting in myocardial ischemia, hypoxia or necrosis caused by heart disease, high disability rate, high recurrence rate, high mortality, complications, and large social burden. Typical acute myocardial infarction (AMI) is a serious coronary heart disease. It is a clinical syndrome caused by myocardial cell ischemia and necrosis due to coronary artery occlusion or severe stenosis. Ami is the leading cause of death and disability. Its symptoms should belong to the TCM chest numbness, true pain and other diseases.

Post-myocardial infarction depression (PMID) belongs to traditional Chinese medicine “sincere pain” or “chest Bi” and “depression syndrome” co-morbidity, co-morbidity, is the basis of coronary heart disease myocardial infarction and depression, due to qi stagnation, blood stasis, phlegm obstruction, fire depression, Y in injury and other comprehensive causes of qi stagnation, liver qi loss of its line, God loss and mood depression, depression, with co-morbidity, co-morbidity characteristics.

With the aging of China's population, the number of patients with coronary heart disease is increasing year by year, and there are many complications left after MI, among which post-MI depression is a common complication, which seriously affects the rehabilitation effect and the quality of life of patients. The disease is mainly heart dysfunction and cognitive dysfunction occur at the same time, mild affect the outcome of myocardial infarction rehabilitation, serious people will have a tendency to despair, and even lead to suicide, if the treatment is not timely, the condition will be light to serious, bringing a heavy burden to the family and society.

PMID is a kind of emotional disorder characterized by low mood, decreased activity ability and retardation of thinking function in addition to various symptoms of coronary heart disease and myocardial infarction after the occurrence of cardiovascular disease. It is a secondary depression and the most common and important complication after coronary heart disease and myocardial infarction.

Depression is a very common mental disorder, and PMID is a common complication after myocardial infarction, a special type of depression, not a simple combination of myocardial infarction and depression, can not be mixed with depression-induced myocardial infarction. Feng Limin et al. found that the combined prevalence of myocardial infarction and depression in Asian patients was 45.03%; Hasham Saeed et al. found that the prevalence rate of myocardial infarction combined with mild, moderate and severe depression was as high as 79.5%, while that of patients combined with mild and moderate depression was about 60%. One meta-analysis showed that post-myocardial infarction depression was associated with a 2-2.5-fold increased risk of cardiovascular complications. A cross-sectional survey of 435 CVD patients by chance sampling and snowball method in the southeastern region of China from October to November 2020 showed that the incidence of anxiety was 11.72% and the incidence of depression was 9.20%. Leong et al. conducted a prospective cohort study that assessed 95 patients for clinical depression and found that 88.4% of patients had depressive symptoms. A study on the prevalence of depression after myocardial infarction showed that 64.3% of patients had varying degrees of emotional problems, of which 66.1%, 23.6%, and 10.2% were mild, moderate, and severe patients, respectively. Therefore, how to safely and effectively treat post-myocardial infarction depression has attracted more and more attention.

At present, Western medicine has a clear identification method for post-myocardial infarction depression, and Western medicine suggests that a combination of psychological therapy, drug therapy and rehabilitation training should be applied to PMID in order to achieve the best therapeutic effect. In terms of drug therapy, compliance, efficacy, adverse reactions, symptom changes, etc. should be monitored and evaluated. Chinese Expert Consensus on Psychological Prescriptions for Patients treated in cardiovascular Department proposes that the use of cardiovascular drugs should be accompanied by anti-anxiety or antidepressant drugs, such as selective 5-HT reuptake inhibitors, benzodiazepines, tricyclic and tetracyclic drugs, monoamine oxidase inhibitors, etc. However, most psychiatric drugs have adverse reactions. The common adverse reactions of selective 5-HT reuptake inhibitors are gastrointestinal symptoms, such as nausea, vomiting and diarrhea, as well as agitation, sexual dysfunction, migraine and weight gain. The common adverse reactions of benzodiazepines are dizziness, lethargy, fatigue and lethargy, and large doses may cause ataxia, coma and respiratory depression. Tricyclic antidepressants often have dry mouth, constipation, blurred vision, dysuria, postural hypotension and other adverse reactions. PMID made recommendations for antidepressant treatment, with citalopram, Sertraline and escitalopram having Level 1 evidence and receiving Level A recommendations (1/A). Caution: Contraindicated in all patients who are allergic to SSRIs or taking monoamine oxidase inhibitors (MAOIs). Use with caution in patients with epilepsy and active intracranial hemorrhage.

In conclusion, the current western treatment is a combination of risk factors (such as epilepsy, falls and delirium) and drug adverse reactions to choose antidepressants. However, western medicine has obvious toxic and side effects, high price, difficult for patients to bear, poor treatment compliance, efficacy needs to be improved and other problems are still difficult to solve. Especially in the clinical treatment practice, about 80% of mild to moderate PMID patients and their families are not willing to take or take anti-depressant western drugs too early, hoping to treat PMID through traditional Chinese medicine.

Traditional Chinese medicine in the treatment of post-MI depression has obvious advantages of overall differentiation of disease and syndrome, and no obvious side effects, patients are easy to accept traditional Chinese medicine treatment. However, there is no new Chinese medicine for PMID at present. In order to facilitate clinical use and improve clinical efficacy, it is urgent to develop new Chinese medicine for PMID.

### SUMMARY

Aiming at the problems existing in the prior art, the first aspect of the design of the invention is to provide a Chinese medicine composition for treating post-coronary heart disease depression, which contains the following components in weight:

1,500-1,600 servings of Ostreae concha, 700-800 servings of Salviae miltiorrhizae radix et rhizoma, 600-700 servings of Forsythiae fructus, 400-500 servings of Dalbergiae odoriferae lignum, 600-700 servings of Ophiopogonis radix, 400-500 servings of Sappan lignum, 10-11 servings of Borneolum syntheticum.

The second aspect of the invention is to provide a preparation method of a Chinese medicine composition for the treatment of post-coronary depression, comprising the following steps:
1) Take the prescribed amount of Forsythiae fructus and Dalbergiae odoriferae lignum, soak them in water, extract with steam distillation, collect the volatile oil, and coat the volatile oil and borneol with β-cyclodextrin to obtain the β-cyclodextrin-coated volatile oil and Borneolum syntheticum for separate use. After centrifuging and removing impurities from the extracted liquid, collect the residue and extract respectively for separate use.
2) Take the amount of Ostreae concha formula and add water to boil, start with the liquid boiling time, fry for 1 h, to obtain the liquid;
3) Step 2) After decocting, add the formula amount of Salviae miltiorrhizae radix et rhizoma, Sappan lignum, Ophiopogonis radix and the residue of step 1) to the decocting solution, reflux extraction, reflux extraction solution, centrifuge separation and purification of the reflux extraction solution, combine with the
extraction solution of step 1), concentrate under pressure, and obtain clear paste;
4) Add the volatile oil and borneol wrapped in β-cyclodextrin prepared in step 1 into the clear paste, spray dry, obtain dry powder, and then add auxiliary materials to obtain particles by dry method.

Further, in step 1), the amount of water added is 8 times the total amount of Forsythiae fructus and Dalbergiae odoriferae lignum, the soaking time is 0.5 hours, and steam distillation is carried out for 7 hours.

Further, in step 2), the solid-liquid ratio of Ostreae concha to water was 1 g:30 L.

Further, in step 3), reflux extraction was carried out twice, each time for 0.5 h, the temperature of decompression concentration was 50-60° C., and the relative density of the clear paste was 1.10±0.02.

The third aspect of the invention is to provide the application of a Chinese medicine composition in the preparation of drugs for the treatment of post-coronary heart disease depression.

The Chinese medicine composition of the invention is suitable for the treatment of depression after myocardial infarction, can improve heart function, restore cognitive impairment, reduce negative emotion, stabilize the condition and improve the quality of life.

### BRIEF DESCRIPTION OF THE DRAWINGS

FIG. 1 is the schematic diagram of AMI model in animal experiments.

FIG. 2 shows the HAMD scores of each group before and after treatment.

FIG. 3 shows the comparison of clinical application results in each group after 4 weeks of treatment.

FIG. 4 shows the comparison of clinical application results in each group after 8 weeks of treatment.

FIG. 5 shows the average score of TCM syndrome before and after treatment of clinical application cases;

FIG. 6 shows the standard score of SCL-90 factor in each group before and after treatment.

FIG. 7 shows the 6-minute walking distance of each group before and after treatment.

FIG. 8 shows the frequency of adverse reactions in clinical applications.

### DESCRIPTION OF THE EMBODIMENTS

The invention is further explained in combination with specific embodiments below in order to better understand the technical scheme.

### Example 1: Guanxin Jieyu Granule (1000 g Granule) Prescription Composition

Ostreae concha 1562.9 g, Salviae miltiorrhizae radix et rhizoma 781.5 g, Forsythiae fructus 625.2 g, Dalbergiae odoriferae lignum 468.9 g, Ophiopogonis radix 625.2 g, Borneolum syntheticum 10.4 g, Sappan lignum 468.9 g, with the addition of auxiliary materials, made into 1000 g granules.

The specific preparation is:
1) Take Forsythiae fructus and Dalbergiae odoriferae lignum and use 8 times the amount of water as the solvent. Soak for 0.5 hours, then distill with steam for 7 hours. Extract and collect the volatile oil, coat the volatile oil and Borneolum syntheticum with β-cyclodextrin, and set them aside for future use. And the medicinal liquid can be extracted. After centrifugation and impurity removal of the extract, it should be kept separately for future use.
2) Take Ostreae concha with 30 times the ratio of feed to liquid water as the solvent, start with the liquid boiling time, fry for 1 h.
3) After the Ostreae concha are boiled, add the residue extracted from Salviae miltiorrhizae radix et rhizoma, Sappan lignum, Ophiopogonis radix and the volatile oil of Forsythiae fructus and Dalbergiae odoriferae lignum. Reflux extract twice, each time for 0.5 hours. The extract is separated by centrifugation to remove impurities and then combined with the volatile oil extract of Forsythiae fructus and Dalbergiae odoriferae lignum. Concentrate under reduced pressure at 50-60° C. to form a clear paste with a relative density of 1.10±0.02.
4) Add the volatile oil and Borneolum syntheticum wrapped in β-cyclodextrin into the clear paste, spray dry, get dry powder, then add maltodextrin and magnesium stearate, dry granule.
Verify eg. 1

Volatile oil extraction experiment: According to the 2020 Edition of the Chinese Pharmacopoeia, the medicinal materials for which volatile oil extraction is required in the present invention are Forsythiae fructus and Dalbergiae odoriferae lignum.

Experimental investigation on the extraction of volatile oil alone and mixed extraction: According to the prescribed dosage, the content of volatile oil extracted from Forsythiae fructus and Dalbergiae odoriferae lignum alone and from the mixed extraction of the two herbs was compared respectively. The results are shown in Table 1.

TABLE 1

| | Volatile Oil | | |
|---|---|---|---|
| | Content | Inventory Rating | Content |
| Drug | (mL) | (g) | (%) |
| Forsythiae Fructus | 0.35 | 20 | 1.75 |
| Dalbergiae | 0.30 | 20 | 1.50 |
| Odoriferae Lignum | | | |
| F + D | 0.60 | 40 | 1.50 |

As can be seen from Table 1, in terms of the amount of volatile oil extracted, there is no significant difference between the total amount extracted when the two drugs are mixed and the total amount extracted separately. To facilitate industrial production, the volatile oil was extracted by mixing Forsythiae fructus and Dalbergiae odoriferae lignum.

Determination of volatile oil extraction method: The present invention studies the extraction process of volatile oils from Forsythiae fructus and Dalbergiae odoriferae lignum. Currently, the main methods used are steam distillation and supercritical fluid extraction. Therefore, it is necessary to compare the extraction efficiencies of these two methods

According to the prescription, 2 pieces of the two herbs were taken and extracted by steam distillation and CO₂ supercritical fluid extraction, respectively.

The results showed that the extraction rate of CO₂ supercritical fluid extraction was higher than that of steam distillation extraction, but there was no significant difference in statistics. Considering the actual production, steam distillation extraction was selected.

Study on the optimum process of extracting volatile oil from two kinds of medicinal materials by steam distillation: The medicinal materials used in the investigation of the volatile oil extraction process were 24 g of Forsythiae fructus and 18 g of Dalbergiae odoriferae lignum. Two factors, extraction time (A) and the ratio of material to liquid (B), were selected, and the yield of volatile oil (C) was taken as the index for optimization. The experiment adopted a 22-factorial design, repeating two blocks. The experimental combinations are shown in Table 2:

TABLE 2
Factorial experiment factors and level table
of volatile oil extraction process

| | | | A | B | C |
|---|---|---|---|---|---|
| | | | Time | solid-liquid | volume |
| std | block | run | (h) | ratio (time) | (mL) |
| 4 | Block 1 | 1 | 7 | 10 | 0.60 |
| 2 | Block 1 | 2 | 7 | 6 | 0.60 |
| 3 | Block 1 | 3 | 4 | 10 | 0.55 |
| 1 | Block 1 | 4 | 4 | 6 | 0.50 |
| 8 | Block 2 | 5 | 7 | 10 | 0.60 |
| 6 | Block 2 | 7 | 7 | 6 | 0.60 |
| 5 | Block 2 | 6 | 4 | 6 | 0.55 |
| 7 | Block 2 | 8 | 4 | 10 | 0.55 |

It can be seen from Table 2 that the model was significant (P<0.05) with the extraction amount of volatile oil as the index. The decoction time had significant difference on the model (P<0.05), and the ratio of material to liquid had no effect. The maximum extraction amount of volatile oil was used as the expected value to obtain the design space and verified by experiments. The experimental conditions were 8 times the ratio of material to liquid and decocted for 7 hours. The verification test results are within the 95% confidence interval.

Therefore, the optimum process was obtained by screening the extraction amount of volatile oil as the index:extraction time 7 h, solid-liquid ratio 1:8.

Encapsulation of volatile oil and borneol β-cyclodextrin (β-CD): Take 0.3 mL of volatile oil, add 0.3 g of Borneolum syntheticum, 2.7 mL of ethanol, and make 3 mL of ethanol solution of volatile oil/Borneolum syntheticum. Take β-CD 3 g, add 30 mL water and stir at 60° C. for 10 min. The β-CD saturated solution was prepared by cooling to room temperature, and then transferred to a constant temperature magnetic stirrer. According to the ratio of β-cyclodextrin to volatile oil (10:1), the ethanol solution of volatile oil/Borneolum syntheticum was added drop by drop under stirring. The magnetic stirring speed was 300 r/min, the temperature was 40° C., and the inclusion was performed for 60 min to a suspension state. After cooling to room temperature, it was refrigerated in a refrigerator for 24 h and then filtered. Washed with anhydrous ethanol for 3 times, the inclusion complex was dried at 40° C. for 5 h to obtain a dry inclusion complex, which was the volatile oil Borneolum syntheticum cyclodextrin inclusion complex.

Study on the optimum extraction process of non-volatile components: The optimum extraction process of non-volatile components The medicinal materials used in the study were a prescription. The extraction time (A), extraction times (B) and solid-liquid ratio (C) were selected as three factors. The water extraction experiment was carried out with the extraction rate (D), forsythiaside A content (E), salvianolic acid B content (F), and forsythin content (G) as response indicators. The 23-factorial design was adopted in the experimental scheme, and the Std1 group repeated two experiments (Std9 and Std10). The experimental combination is shown in Table 3:

TABLE 3
Factorial experiment factors and level table of water extraction process

| | | A | B | C | D | E | F | G |
|---|---|---|---|---|---|---|---|---|
| | | time | Frquency | Soild-liquid | Extract | Lqzga | Db | Lqg |
| std | run | (h) | (time) | ratio (time) | yield | (mg) | (mg) | (mg) |
| 1 | 5 | 0.5 | 1 | 10 | 0.1645 | 437.412 | 296.775 | 21.017 |
| 2 | 4 | 2 | 1 | 10 | 0.1949 | 613.493 | 239.267 | 30.093 |
| 3 | 0 | 0.5 | 3 | 10 | 0.2362 | 810.112 | 374.387 | 28.591 |
| 4 | 1 | 2 | 3 | 10 | 0.2555 | 866.211 | 247.337 | 36.961 |
| 5 | 3 | 0.5 | 1 | 20 | 0.1890 | 485.541 | 302.937 | 23.576 |
| 6 | 10 | 2 | 1 | 20 | 0.2090 | 637.268 | 226.449 | 26.339 |
| 7 | 2 | 0.5 | 3 | 20 | 0.2699 | 794.456 | 310.611 | 24.935 |
| 8 | 8 | 2 | 3 | 20 | 0.2723 | 784.849 | 248.776 | 30.063 |
| 9 | 7 | 0.5 | 1 | 10 | 0.1535 | 380.044 | 267.543 | 19.773 |
| 10 | 6 | 0.5 | 1 | 10 | 0.1845 | 511.413 | 307.857 | 21.012 |

It can be seen from Table 3 that the model of this experiment was significant. The decoction time and decoction times had significant differences in each index (P<0.05), and there was no significant difference in the ratio of material to liquid. The expected values of the four indexes of extract rate, forsythiaside A content, salvianolic acid B content and forsythin content were set as the maximum values, and several optimal processes were obtained. The process with the highest expected value was selected for verification, and the indicators of the three verification tests fell within the 95% confidence interval.

Therefore, the four indexes of extract rate, forsythiaside A content, salvianolic acid B content and forsythin content were screened to obtain the best process:extraction time 0.5 h, solid-liquid ratio 1:10, extraction times 2 times.

### Determination of Optimum Extraction Process Parameters

The water extraction process: Take two medicinal herbs, Forsythiae fructus and Dalbergiae odoriferae lignum, add 8 times the amount of water, extract for 7 hours to obtain the volatile oil, collect the volatile oil, wrap it with β-cyclodextrin and Borneolum syntheticum, and set it aside for future use. Also, obtain the extraction liquid, centrifuge and separate the extraction liquid to remove impurities, and set it aside for future use. Take an appropriate amount of Ostreae concha and add 30 times the amount of water. Extract for 1 hour to obtain the liquid from the Ostreae concha. Then, add Salviae miltiorrhizae radix et rhizoma, Sappan lignum, Ophiopogonis radix and the volatile oil of Forsythiae fructus and Dalbergiae odoriferae lignum to extract the residue. Extract twice, each time for 0.5 hours. Filter the liquid and combine it with the volatile oil extract of Forsythiae fructus and Dalbergiae odoriferae lignum, and concentrate it into a extract for later use.

Separation and impurity removal process: In order to remove the solid particles in the liquid, the factory mostly uses the method of filtration. Because the prescription contains more polysaccharides, the resulting liquid is sticky and difficult to filter. After the experiment, when the liquid is concentrated to about 1.1, the liquid is centrifuged with a tubular centrifuge to obtain a clearer liquid. The loss rate before and after impurity removal was about 5% calculated by salvianolic acid B and forsythin. According to the above experimental results, depending on the liquid and plant equipment conditions in the production can consider the choice of tubular centrifuge centrifugal impurity removal.

Concentration in drying process: Since this process adopts water extraction process, the concentration is carried out under conventional vacuum (≤0.09 mPa) and low temperature (≤60° C.) concentration conditions.

This process uses spray drying to prepare extract powder, and then granulation. Firstly, a single factor experiment was conducted to investigate the influence of the relative density of liquid medicine on the experiment. The results showed that when the density of liquid medicine was 1.05 g/cm3, extractum powder could not be sprayed, and when the density of liquid medicine was 1.10, extractum powder with good properties could be prepared, while when the density of liquid medicine was 1.20 g/cm3, the instrument injection needle would be blocked. Therefore, in this experiment, the density of the liquid was fixed at 1.10 g/cm3, the water content of the extract powder was taken as the evaluation index, and the effects of air inlet temperature and sample injection speed on the drying process were investigated. The spray drying process conditions were experimentally screened according to the factor test table. The results are shown in Table 4.

TABLE 4
Factorial experimental factors and
level table of spray drying process

| | | A | B | C |
|---|---|---|---|---|
| | | intake air temperature | intake velocity | water content |
| std | run | (° C.) | (rpm) | (%) |
| 1 | 8 | 150 | 10 | 3.45 |
| 2 | 4 | 150 | 10 | 3.77 |
| 3 | 7 | 180 | 10 | 4.05 |
| 4 | 5 | 180 | 10 | 2.80 |
| 5 | 3 | 150 | 15 | 5.89 |
| 6 | 6 | 150 | 15 | 5.07 |
| 7 | 2 | 180 | 15 | 3.33 |
| 8 | 1 | 180 | 15 | 3.33 |

As can be seen from Table 4, this test model is significant, and the inlet air temperature factor has a significant difference (P<0.05), while the injection speed factor has no significant influence. According to the design space, all the indexes of the two verification tests fall within the 95% confidence interval.

Therefore, the optimal spray conditions were air inlet temperature 156° C., creep velocity 12 rpm, and relative density of liquid 1.10 g/cm3.

Forming process: The spray drying powder obtained by the above process has strong hygroscopical properties, and it is not suitable to use wet granulation after pre-testing. Because there is no process of humidification and drying, and it has the advantages of low cost and energy saving, the dry granulation process is selected for investigation.

Selection of excipients type and dosage: According to the commonly used excipients of granules, dextrin and maltodextrin were selected as alternative excipients in this experiment, mainly from the particle yield. Add 1% magnesium stearate to prevent sticking rolls.

After dry granulation, the product is placed in 10 mesh and 80 mesh sieve, and the weight between 10~80 mesh sieve is accurately weighed to calculate the yield. The results showed that the average molding rate of maltodextrin was higher than that of dextrin. Considering the price of excipients, the dry granule excipients could be determined as maltodextrin. According to the above experimental results, the mixture of 19% maltodextrin and 1% magnesium stearate was selected as the granulating excipients.

Dry granulation process parameters: Select the dry granulation conditions, the environmental humidity, the main pressure, the amount of added auxiliary materials for a preliminary study, after the pre-test, the environmental relative humidity of 40%, the main pressure of 5 MPa, the amount of 20% auxiliary materials the highest yield, the particle yield of 70%.

### Animal Experiment

### 1. Preparation of Acute Myocardial Infarction (AMI) Model in Rats

The long-term survival model of acute myocardial infarction after coronary ligation was established by anterior descending coronary artery ligation in vivo.

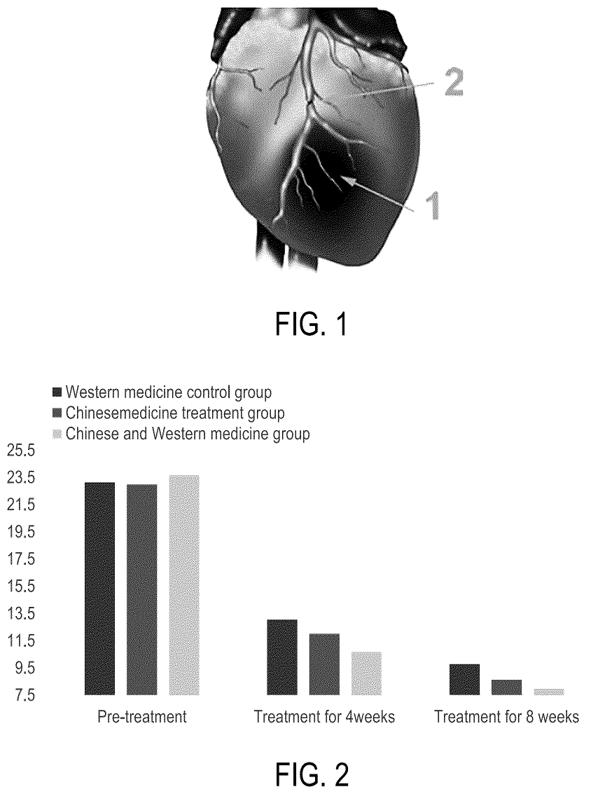

After anesthesia (Suthai 50 combined with atropine), the rats were shaved in the chest with a small animal hair clipper, fixed the posterior tracheal intubation, and connected to the small animal ventilator. The skin was cut along the intercostal direction with a length of about 1.5 cm. The subcutaneous tissue and muscle were separated layer by layer and the pericardium was opened. The anterior descending branch of the left coronary artery was lapped 2 mm below the edge of the auricle with 6.0 sutures. During the operation, the white area of myocardium in the anterior and lateral wall of the left ventricle at the distal end of the ligation line was visible to the naked eye. Appropriate amount of lidocaine was added to prevent arrhythmia, the chest was closed layer by layer, and appropriate amount of penicillin was given to prevent postoperative infection. The ventilator was removed and the rat's chest was gently pinched to assist it to recover breathing. All the above operations were performed under strict aseptic conditions. Keep warm during and after operation before the rats wake up. The operation of Control group was the same as that of AMI model animals except that coronary artery was not ligation. See FIG. 1.

2. Preparation of Depression Model after Myocardial Infarction

On the basis of successful AMI model preparation, on the 4th day after AMI model modeling, all the depressed rats were placed in solitary cages, and combined with CUMS method, two of the following nine methods were randomly selected for stress treatment every day, and were treated continuously for 28 days. The detailed operation method improves Willner P's method and slightly modifies it, as shown in Table 5-Table 6.

TABLE 5

| Method | Specific operation method |
|---|---|
| Fasting | No diet was given for 20 hours each time |
| Water-depriving | Do not give drinking water for 17 hours at a time |
| Shake the rat cage | The rat cage was shaken horizontally for 15 minutes, which |
| | was based on the rat's inability to stand. |
| Day-night reversal | Sunlight was directly irradiated for 17 hours from 5:00 |
| | p.m. to 10:00 a.m. the next day. |
| Wet padding | Put 100 g sawdust into each cage and add 200 mL water for |
| | 21 hours/time. |
| Swimming in ice water | The rats were placed in a plastic bucket with a smooth wall |
| | and a height of about 50 cm in an ice water bucket at 4° C. |
| | The water depth in the bucket was about 15 cm (to the |
| | extent that the toe of the hind foot could just touch the |
| | bottom of the bucket), 5 min times. After completion, the |
| | whole body of the rats was dried with a hairdryer. |
| Odor stimulation | Pepper powder is sprinkled into rat cages for 24 hours |
| Bondage | Using self-made behavior limiter (unified with Nongfu |
| | mountain spring 450 mL plastic bottle cut off the bottom of |
| | the bottle, remove the cap so that the rat's head toward the |
| | mouth of the bottle to climb into the bottle after fixed with |
| | transparent plastic cut off the bottom of the bottle), for 2 |
| | hours/times. |
| Tail clamp | Put the rat in the rat cage, cover the cage cover, and then pull |
| | its tail from the cage cover gap, with a file clip to clamp the |
| | rat tail, (the force should not be too large, so as not to tear |
| | off the rat tail), 5 minutes/time |

TABLE 6
Order of stress method arrangement

| Time | Method |
|---|---|
| Day 1 | Fasting, Shaking cage |
| Day 2 | Water deprivation, Circadian rhythm disruption |
| Day 3 | Damp bedding, Odor stimulation |
| Day 4 | Ice water swimming, Restraint |
| Day 5 | Tail pinch, Fasting |
| Day 6 | Shaking cage, Water deprivation |
| Day 7 | Odor stimulation, Damp bedding |
| Day 8 | Circadian rhythm disruption, Ice water swimming |
| Day 9 | Shaking cage, Tail pinch |
| Day 10 | Restraint, Crowding |
| Day 11 | Fasting, Strobe light |
| Day 12 | Water deprivation, Circadian rhythm disruption |
| Day 13 | Damp bedding, Shaking cage |
| Day 14 | Ice water swimming, Restraint |
| Day 15 | Tail pinch, Fasting |
| Day 16 | Shaking cage, Water deprivation |
| Day 17 | Odor stimulation, Damp bedding |
| Day 18 | Circadian rhythm disruption, Ice water swimming |
| Day 19 | Odor stimulation, Tail pinch |
| Day 20 | Restraint, Shaking cage |
| Day 21 | Fasting, Shaking cage |
| Day 22 | Odor stimulation, Ice water swimming |
| Day 23 | Fasting, Restraint |
| Day 24 | Damp bedding, Tail pinch |
| Day 25 | Odor stimulation, Tail pinch |
| Day 26 | Fasting, Circadian rhythm disruption |
| Day 27 | Ice water swimming, Damp bedding |
| Day 28 | Restraint, Water deprivation |

Grouping and administration: After 24 hours of modeling, the surviving rats will enter the experimental group. According to the random number table method, the rats will be divided into Control group, Model group, Fluoxetine group and FM group. ZY DZ1 group, ZY DZ2 group, ZY DZ3 group, ZY DZ4 group, ZY DZ5 group, ZY DZ5 group, ZY DZ6 group, ZY DZ7 group, ZY DZ8 group, ZY DZ9 group, ZY DZ10 group, ZY DZ11 group, ZY DZ12 group, ZY DZ13 group, ZY DZ14 group, ZY DZ15 group, ZY DZ16 group, ZY DZ17 group, ZY DZ18 group, ZY DZ19 group. Rats in each group were given intragastric administration of equal volume solution, among which, Control group and Model group were given intragastric administration of equal volume normal saline. Continuous 56 d. In this experiment, the dosage of Chinese medicine in each group was converted according to the clinical dosage.
1) Fluoxetine hydrochloride group (0.01 g/kg/d)
2) FM group Guanxin Jieyu Decoction: Salviae miltiorrhizae radix et rhizoma 15 g, Sappan lignum 9 g, Ostreae concha 30 g, Dalbergiae odoriferae lignum 9 g, Forsythiae fructus 12 g, Ophiopogonis radix 12 g, Borneolum syntheticum 0.25 g.
3) ZY DZ1 group (Application number: CN201710062005.0): Fossilia Ossis Mastodi 20 g, Curcumae radix 10 g, Forsythiae fructus 10 g, Asparagi radix 10 g, Scrophulariae radix 12 g, Rehmanniae radix cruda 12 g, Ophiopogonis radix 10 g, Natrii sulfas exsiccatus 12 g, Aquilariae lignum resinatum 5 g, Haliotidis concha praeparata 10 g, Micae lapis aureus 15 g, Citri reticulatae pericarpium 10 g, Fel suillus recens 30 g, Poria 10 g, Salviae miltiorrhizae radix et rhizoma 12 g, Glycyrrhizae radix et rhizoma 7 g.
4) ZY DZ2 group (Application number: CN202210407573.0): Astragali radix 15 g, Codonopsis radix 15 g, Ophiopogonis radix 12 g, Polygoni multiflori radix 9 g, Epimedii folium 9 g, Puerariae lobatae radix 12 g, Angelicae sinensis radix 9 g, Salviae miltiorrhizae radix et rhizoma 15 g, Gleditsiae spina 8 g, Sargassum 9 g, Thallus eckloniae 9 g, Ostreae concha 15 g, Aurantii fructus immaturus 9 g.
5) ZY DZ3 group (Application number: CN202111612514.9): Notoginseng radix et rhizoma 5 g, Astragali radix 12 g, Salviae miltiorrhizae radix et rhizoma 12 g, Trichosanthis fructus 15 g, Curcumae radix 9 g, Dalbergiae odoriferae lignum 9 g, Corydalis rhizoma 12 g, Aurantii fructus immaturus 9 g, Choerospondiatis fructus 5 g, Rosae laevigatae fructus 6 g, Ophiopogonis radix 9 g, Ziziphi spinosae semen 30 g, Asari radix et rhizoma 3 g, Borneolum syntheticum 0.2 g, Moschus 5 g, Benzoinum 9 g.
6) ZY DZ4 group (Application number: CN201810606242.3): Aquilariae lignum resinatum 5 g, Carthami flos 9 g, Ginseng radix et rhizoma 6 g, Ophiopogonis radix 9 g, Schisandrae chinensis fructus 10 g, Notoginseng radix et rhizoma 3 g, Angelicae sinensis radix 9 g, Salviae miltiorrhizae radix et rhizoma 9 g, Chuanxiong rhizoma 8 g, Curcumae radix 6 g, Olibanum 9 g, Draconis sanguis 6 g, Astragali radix 12 g, Glycyrrhizae radix et rhizoma 8 g, Succinum 9 g, Acori tatarinowii rhizoma 10 g, Cinnabaris 3 g, Dalbergiae odoriferae lignum 9 g, Styrax 5 g, Benzoinum 5 g, Santali albi lignum 6 g, Borneolum syntheticum 0.4 g
7) ZY DZ5 group (application number: CN201410059638.2): Salviae miltiorrhizae radix et rhizoma 9 g, Astragali radix 12 g, Corydalis rhizoma 10 g, Ophiopogonis radix 1 g, Chuanxiong rhizoma 9 g, Curcumae radix 12 g, Citri reticulatae pericarpium viride 6 g, Spatholobi caulis 10 g, Crataegi fructus 15 g, Trichosanthis fructus 15 g, Ginseng radix et rhizoma 8 g, Persicae semen 6 g, Crocus sativus 3 g, Paeoniae radix rubra 6 g, Cyperi rhizoma 9 g, Aurantii fructus 15 g, Citri reticulatae pericarpium 9 g, Glycyrrhizae radix et rhizoma 8 g, Angelicae sinensis radix 12 g, Bupleuri radix 8 g, Dalbergiae odoriferae lignum 12 g, Amomi fructus 3 g, Acori tatarinowii rhizoma 6 g, Trogopterori faeces 5 g, Polygonati odorati rhizoma 9 g, Notoginseng radix et rhizoma 3 g, Jasmini flos 6 g, Santali albi lignum 3 g, Borneolum syntheticum 0.2 g.
8) ZY DZ6 group (Application number: CN201610833974.7): Dalbergiae odoriferae lignum 9 g, Paeoniae radix rubra 6 g, Santali albi lignum 2 g, Salviae miltiorrhizae radix et rhizoma 8 g, Angelicae sinensis radix 8 g, Curcumae radix 8 g, Chuanxiong rhizoma 5 g, Ophiopogonis radix 10 g, Ganoderma 6 g, Hirudo 3 g, Styrax 3 g, Borneolum syntheticum 0.15 g.
9) ZY DZ7 group (Application number: CN201610017979.2): Sargassum 10 g, Thallus eckloniae 10 g, Salviae miltiorrhizae radix et rhizoma 10 g, Ostreae concha 10 g, Poria 5 g, Chuanxiong rhizoma 5 g, Angelicae sinensis radix 5 g, Paeoniae radix rubra 5 g, Pinelliae rhizoma praeparatum 5 g, Citri reticulatae pericarpium 5 g, Carthami flos 5 g, Sappan lignum 5 g, Dalbergiae odoriferae lignum 5 g.
10) ZY DZ8 group (Application number: CN201610584682.4): Panacis quinquefolii radix 6 g, Salviae miltiorrhizae radix et rhizoma 15 g, Spatholobi caulis 9 g, Glehniae radix 12 g, Ophiopogonis radix 9 g, Rhodiolae crenulatae radix et rhizoma 9 g, Camelliae sinensis radix 3 g, Notoginseng radix et rhizoma 8 g, Paeoniae radix rubra 12 g, Chuanxiong rhizoma 15 g, Dalbergiae odoriferae lignum 12 g, Corydalis rhizoma 15 g, Curcumae radix 8 g, Sophorae flavescentis radix 6 g, Gentianae macrophyllae radix 6 g, Borneolum syntheticum 0.3 g.
11) ZY DZ9 group (Application number: CN201410059640.X): Salviae miltiorrhizae radix et rhizoma 9 g, Ginkgo folium 12 g, Corydalis rhizoma 15 g, Chuanxiong rhizoma 18 g, Hordei fructus germinatus 12 g, Moutan cortex 9 g, Crataegi fructus 9 g, Ginseng radix et rhizoma 3 g, Persicae semen 6 g, Crocus sativus 3 g, Paeoniae radix rubra 8 g, Spatholobi caulis 12 g, Aurantii fructus 9 g, Citri reticulatae pericarpium 9 g, Artemisiae anomalae herba 3 g, Glycyrrhizae radix et rhizoma 5 g, Angelicae sinensis radix 12 g, Rehmanniae radix cruda 9 g, Bupleuri radix 15 g, Dalbergiae odoriferae lignum 12 g, Paeoniae radix alba 15 g, Allii macrostemonis bulbus 8 g, Platycodonis radix 9 g, Tetrapanacis medulla 5 g, Trogopterori faeces 5 g, Cinnamomi ramulus 3 g, Polygonati odorati rhizoma 9 g, Notoginseng radix et rhizoma 3 g, Styrax 3 g, Borneolum syntheticum 0.2 g.
12) ZY DZ10 group (Application number: CN201510604049.2): Panacis quinquefolii radix 30 g, Salviae miltiorrhizae radix et rhizoma 30 g, Glehniae radix 30 g, Sophorae flavescentis radix 30 g, Notoginseng radix et rhizoma 30 g, Ophiopogonis radix 30 g, Paeoniae radix rubra 50 g, Chuanxiong rhizoma 30 g, Dalbergiae odoriferae lignum 50 g, Gentianae macrophyllae radix 30 g, Borneolum syntheticum 15 g.
13) ZY DZ11 group (Application number: CN201410373829.6): Codonopsis radix 15 g, Glycyrrhizae radix et rhizoma 8 g, Cistanches herba 6 g, Astragali radix 15 g, Lygodii rhizoma 9 g, Sparganii rhizoma 9 g, Carthami flos 9 g, Crataegi cuneatae fructus 9 g, Salviae miltiorrhizae radix et rhizoma 9 g, Borneolum syntheticum 0.2 g, Chuanxiong rhizoma 15 g, Cinnamomi cortex 12 g, Astragali complanati semen 9 g, Akebiae caulis 8, Paeoniae radix rubra 12 g, Rehmanniae radix praeparata 8 g, Angelicae sinensis radix 8 g, Lophatheri herba 9 g, Oryzae sativae germinatus fructus 8 g, Eupatorii lindleyani herba 12 g, Ophiopogonis radix 15 g, Platycladi semen 15 g.
14) ZY DZ12 group (Application number: CN201310110797.6): Panacis quinquefolii radix 2 g, Astragali radix 20 g, Crocus sativus 2 g, Salviae miltiorrhizae radix et rhizoma 20 g, Trichosanthis fructus 20 g, Curcumae radix 6 g, Hordei fructus germinatus 20 g, Dalbergiae odoriferae lignum 2 g, Ophiopogonis radix 6 g, Borneolum syntheticum 0.05 g
15) ZY DZ13 group (Application number: CN201410059637.8): Salviae miltiorrhizae radix et rhizoma 12 g, Astragali radix 9 g, Corydalis rhizoma 8 g, Chuanxiong rhizoma 8 g, Curcumae radix 6 g, Ophiopogonis radix 9 g, Spatholobi caulis 15 g, Crataegi fructus 8 g, Curcumae longae rhizoma 9 g, Ginseng radix et rhizoma 6 g, Sparganii rhizoma 8 g, Crocus sativus 8 g, Paeoniae radix rubra 3 g, Polygonati rhizoma 12 g, Cinnamomi ramulus 8 g, Citri reticulatae pericarpium 3 g, Glycyrrhizae radix et rhizoma 8 g, Angelicae sinensis radix 5 g, Cistanches herba 15 g, Dalbergiae odoriferae lignum 9 g, Rhodiolae crenulatae radix et rhizoma 15 g, Acori tatarinowii rhizoma 9 g Trogopterori faeces 6 g, Polygonati odorati rhizoma 9 g, Notoginseng radix et rhizoma 15 g, Rhei radix et rhizoma 8 g, Borneolum syntheticum 0.2 g.
16) ZY DZ14 group (Application number: CN200410020621.2): Ginseng radix et rhizoma 3 g, Ophiopogonis radix 9 g, Schisandrae chinensis fructus 15 g, Astragali radix 12 g, Dioscoreae rhizoma 9 g, Atractylodis rhizoma 9 g, Scrophulariae radix 12 g, Coptidis rhizoma 8 g, Puerariae lobatae radix 8 g, Salviae miltiorrhizae radix et rhizoma 18 g, Crataegi fructus 8 g, Rehmanniae radix cruda 15 g, Trichosanthis radix 20 g, Paeoniae radix rubra 15 g, Dalbergiae odoriferae lignum 15 g, Borneolum syntheticum 0.3 g.
17) ZY DZ15 group (Application number: CN200610010165.2): Ginseng radix et rhizoma 8 g, Ophiopogonis radix 8 g, Schisandrae chinensis fructus 8 g, Notoginseng radix et rhizoma 8 g, Angelicae sinensis radix 10 g, Salviae miltiorrhizae radix et rhizoma 16 g, Chuanxiong rhizoma 12 g, Curcumae radix 8 g, Olibanum 4 g, Draconis sanguis 4 g, Astragali radix 20 g, Glycyrrhizae radix et rhizoma 8 g, Succinum 8 g, Acori tatarinowii rhizoma 4 g, Cinnabaris 2 g, Dalbergiae odoriferae lignum 4 g, Styrax 6 g, Benzoinum 4 g, Santali albi lignum 4 g, Borneolum syntheticum 2 g.
18) ZY DZ16 group (Application No.: CN01131203.3): Ginseng radix et rhizoma 15 g, Codonopsis radix 12 g, Pseudostellariae radix 15 g, Astragali radix 9 g, Atractylodis macrocephalae rhizoma 12 g, Glycyrrhizae radix et rhizoma 9 g, Dioscoreae rhizoma 8 g, Hirudo 12 g, Salviae miltiorrhizae radix et rhizoma 15 g, Chuanxiong rhizoma 9 g, Carthami flos 8 g, Paeoniae radix rubra 12 g, Dalbergiae odoriferae lignum 9 g, Notoginseng radix et rhizoma 6 g, Curcumae radix 8 g, Persicae semen 15 g, Corydalis rhizoma 15 g, Eupolyphaga 9 g, Olibanum 9 g, Draconis sanguis 8 g, Myrrha 9 g, Spatholobi caulis 15 g, Aquilariae lignum resinatum 8 g, Zedoariae rhizoma 9 g, Sparganii rhizoma 9 g, Leonuri herba 9 g, Trogopterori faeces 15 g, Achyranthis bidentatae radix 9 g, Trichosanthis fructus 15 g, Pinelliae rhizoma 9 g, Allii macrostemonis bulbus 15 g, Armeniacae semen amarum 9 g, Aurantii fructus 15 g, Aurantii fructus immaturus 9 g, Santali albi lignum 9 g, Cyperi rhizoma 15 g, Aucklandiae radix 9 g, Citri reticulatae pericarpium 9 g, Platycodonis radix 9 g, Corydalis rhizoma 15 g, Polygoni multiflori radix 9 g, Angelicae sinensis radix 9 g, Rehmanniae radix praeparata 9 g, Paeoniae radix alba 15 g, Ophiopogonis radix 15 g, Polygonati rhizoma 9 g, Borneolum syntheticum 0.2 g, Acori tatarinowii rhizoma 9 g, Ziziphi spinosae semen 15 g, Polygalae radix 9 g, Succinum 8 g, Asari radix et rhizoma 9 g, Aconiti lateralis radix praeparata 15 g, Cinnamomi ramulus 8 g, Epimedii herba 9 g, Schisandrae chinensis fructus 9 g, Crataegi fructus 15 g, Puerariae lobatae radix 15 g, Poria 9 g, Angelicae dahuricae radix 6 g, Bupleuri radix 9 g, Lumbricus 15 g, Gastrodiae rhizoma 9 g, Scorpio 9 g, Cicadae periostracum 8 g, Scolopendra 8 g.
19) ZY DZ17 group (Application number: CN201510009879.0): Astragali radix 20 g, Pseudostellariae radix 20 g, Polygalae radix 10 g, Ziziphi spinosae semen 15 g, Aucklandiae radix 10 g, Rehmanniae radix cruda 10 g, Ophiopogonis radix 10 g, Lycii fructus 15 g, Glycyrrhizae radix et rhizoma praeparata 10 g, Corni fructus 12 g, Asini corii colla 10 g, Salviae miltiorrhizae radix et rhizoma 10 g, Puerariae lobatae radix 10 g, Achyranthis bidentatae radix 10 g, Dalbergiae odoriferae lignum 10 g.
20) ZY DZ18 group (Chen Chunlei, Cheng Xiaoyu. Cheng Xiaoyu merger experience anxiety depression treatment of the coronary heart disease [J]. Journal of traditional Chinese medicine clinical journal, 2024, 4 (8):1460-1464. The DOI: 10.16448/j.carol carroll JTCM.2024.0810): Astragali radix 15 g, Codonopsis radix 15 g, Trichosanthis fructus 15 g, Salviae miltiorrhizae radix et rhizoma 15 g, Angelicae sinensis radix 15 g, Chuanxiong rhizoma 15 g, Paeoniae radix rubra 15 g, Citri reticulatae pericarpium 9 g, Curcumae radix 15 g, Dalbergiae odoriferae lignum 15 g, Ophiopogonis radix 15 g, Glycyrrhizae radix et rhizoma praeparata 8 g.
21) ZY DZ19 group (Gu Yanpin, Tao Danhong. Based on the theory of ‘heart governing mind’, the effect of Yixin Jieyu Decoction on mild depression after heart failure was studied [J]. Chinese Medical Herald, 2017, 14 (7):73-76): Salviae miltiorrhizae radix et rhizoma 20 g, Curcumae radix 15 g, Trichosanthis fructus et pericarpium 8 g, Allii macrostemonis bulbus 18 g, Dalbergiae odoriferae lignum 9 g, Margaritifera concha 30 g, Ophiopogonis radix 15 g, Rehmanniae radix praeparata 40 g, Polygalae radix 9 g, Albiziae cortex 8 g, Polygoni multiflori caulis 15 g, Aurantii fructus 15 g, Citri reticulatae pericarpium 8 g, Platycodonis radix 8 g, Poria 9 g, Alismatis rhizoma 8 g, Cinnamomi ramulus 15 g.

### 3. Testing Index

$$Body weight : The body weight of rats was measured before administration and 56 days after administration , and the average body weight growth rate was calculated = [ ( average body weight after administration - average body weight before administration ) / average body weight before administration ] \times 100 \%$$

### Behavioral Experiment

Sugar Water Preference Experiment: Rats were placed in a single mouse box, and two weighed water bottles (1% sugar water and pure water respectively) were placed on each mouse box. After the rats were deprived of water for 24 hours, the percentage of sugar water preference of rats within 6 hours [(sugar water consumption/total liquid consumption)×100%] was used as the evaluation index. The sugar water preference test was performed on the 56th day after administration.

Open Field Test: The rats were placed in the center of the self-made open box, adjusted for 2 minutes, and the total distance of the rats' movement within 5 minutes was recorded. Tests were performed before and on the 56th day after administration.

Detection of neurotransmitters in rat brain tissue: The cortex and hippocampus were separated from the removed rat brain tissue, and the hippocampus tissue of the rat was mixed with 1:3 normal saline in the ice bath. After centrifuging, the supernatant was obtained. The contents of serotonin (5-HT), dopamine (DA) and norepinephrine (NE) were detected by ELSIA method, and the concentration of neurotransmitter was converted.

### Test Situation and Results

Effect of the invention on the body mass of rats: compared with the model group, the invention group can significantly improve the state of body mass decline caused by depression after myocardial infarction (P<0.05). See Table 7.

TABLE 7
Influence of the invention on the body mass of rats

| | Quality of | | Average body |
|---|---|---|---|
| | predrug | Body mass after | mass growth |
| Group | delivery (g) | administration (g) | rate (%) |
| Control | 231.0 ± 1.8 | 514.1 ± 10.2 | 122.6 |
| Model | 230.7 ± 3.6 | 480.3 ± 5.5* | 108.2 |
| Fluoxetine | 230.2 ± 2.8 | 497.0 ± 8.1^{#} | 115.9 |
| FM | 231.6 ± 3.1 | 491.5 ± 10.7^{#} | 112.2 |
| ZYDZ1 | 231.9 ± 5.1 | 488.0 ± 8.5 | 110.5 |
| ZYDZ2 | 230.5 ± 6.6 | 488.2 ± 9.7 | 111.8 |
| ZYDZ3 | 230.5 ± 4.4 | 486.9 ± 8.9 | 111.2 |
| ZYDZ4 | 230.4 ± 4.3 | 490.0 ± 6.9^{#} | 112.7 |
| ZYDZ5 | 231.0 ± 3.0 | 484.4 ± 9.0 | 109.7 |
| ZYDZ6 | 231.3 ± 3.8 | 486.9 ± 8.3 | 110.6 |
| ZYDZ7 | 229.8 ± 3.9 | 486.3 ± 10.3 | 111.7 |
| ZYDZ8 | 229.7 ± 4.4 | 488.4 ± 10.5^{#} | 112.6 |
| ZYDZ9 | 230.6 ± 5.9 | 489.9 ± 9.9^{#} | 112.4 |
| ZYDZ10 | 230.7 ± 3.4 | 486.9 ± 7.6 | 111.0 |
| ZY DZ11 | 231.7 ± 4.7 | 486.2 ± 9.9 | 109.9 |
| ZY DZ12 | 230.8 ± 3.7 | 486.6 ± 8.6 | 110.9 |
| ZY DZ13 | 230.5 ± 5.7 | 485.8 ± 13.6 | 110.7 |
| ZY DZ14 | 230.9 ± 3.7 | 485.4 ± 9.3 | 110.3 |
| ZY DZ15 | 231.0 ± 3.9 | 490.6 ± 10.9^{#} | 112.3 |
| ZY DZ16 | 231.3 ± 4.0 | 486.6 ± 8.1 | 110.4 |
| ZY DZ17 | 231.7 ± 3.8 | 488.7 ± 10.0^{#} | 110.9 |
| ZY DZ18 | 232.0 ± 4.4 | 489.4 ± 10.4^{#} | 111.0 |
| ZY DZ19 | 231.2 ± 6.5 | 489.5 ± 8.6^{#} | 111.7 |

Note:
Compared with the control group, *P < 0.05;
compared with the model group, ^{#}P < 0.05.

The effect of the invention on the preference of sugar water in rats: after 56 days of administration, compared with the model group, the invention group can significantly improve the preference rate of sugar water in the model group (P<0.05). See Table 8.

TABLE 8
Effect of the invention on the sugar water preference rate of rats

| Group | Sugar water preference (%) |
|---|---|
| Control | 70.5 ± 3.0 |
| model | 46.7 ± 5.3* |
| fluoxetine | 63.0 ± 2.9^{#} |
| FM | 59.3 ± 2.8^{#} |
| ZYDZ1 | 49.4 ± 7.5 |
| ZYDZ2 | 48.5 ± 6.0 |
| ZYDZ3 | 60.6 ± 3.9^{#} |
| ZYDZ4 | 50.5 ± 5.5 |
| ZYDZ5 | 49.1 ± 5.3 |
| ZYDZ6 | 46.6 ± 8.3 |
| ZYDZ7 | 49.1 ± 6.7 |
| ZYDZ8 | 60.1 ± 3.1^{#} |
| ZYDZ9 | 49.6 ± 3.7 |
| ZYDZ10 | 46.1 ± 4.8 |
| ZY DZ11 | 48.2 ± 3.8 |
| ZY DZ12 | 59.1 ± 1.9^{#} |
| ZY DZ13 | 48.5 ± 3.4 |
| ZY DZ14 | 48.0 ± 6.9 |
| ZY DZ15 | 51.2 ± 4.3 |
| ZY DZ16 | 49.3 ± 3.6 |
| ZY DZ17 | 50.1 ± 3.8 |
| ZY DZ18 | 59.7 ± 3.2^{#} |
| ZY DZ19 | 48.6 ± 5.6 |

Note:
Compared with control group, *P < 0.05;
Compared with model group, ^{#}P < 0.05.

The effect of the invention on the total movement distance of rats is as follows: compared with the model group, the total movement distance of rats in the model group can be significantly increased in the invention group after administration for 56 days (P<0.05). See Table 9.

TABLE 9
Effect of the invention on the total distance of the rat movement

| Group | Total distance of motion (m) |
|---|---|
| control | 21.2 ± 2.2 |
| model | 6.9 ± 1.4* |
| fluoxetine | 10.4 ± 2.3^{#} |
| FM | 11.0 ± 1.4^{#} |
| ZYDZ1 | 10.2 ± 3.8^{#} |
| ZYDZ2 | 7.6 ± 2.1 |
| ZYDZ3 | 8.0 ± 1.6 |
| ZYDZ4 | 10.9 ± 2.5^{#} |
| ZYDZ5 | 7.5 ± 1.6 |
| ZYDZ6 | 7.6 ± 1.7 |
| ZYDZ7 | 10.6 ± 2.1^{#} |
| ZYDZ8 | 7.5 ± 0.8 |
| ZYDZ9 | 7.4 ± 1.4 |
| ZYDZ10 | 11.1 ± 2.7^{#} |
| ZY DZ11 | 7.6 ± 1.5 |
| ZY DZ12 | 7.8 ± 1.1 |
| ZY DZ13 | 12.7 ± 1.4^{#} |
| ZY DZ14 | 7.0 ± 1.6 |
| ZY DZ15 | 8.2 ± 1.9 |
| ZY DZ16 | 12.6 ± 1.4^{#} |
| ZY DZ17 | 7.6 ± 1.8 |
| ZY DZ18 | 7.8 ± 1.1 |
| ZY DZ19 | 7.2 ± 1.1 |

Note:
Compared with control group, *P < 0.05;
Compared with model group, ^{#}P < 0.05.

The effect of the invention on neurotransmitters in rat brain tissue: on the 56th day, compared with the Control group, the contents of 5-HT, DA and NE in rat brain tissue in Model group were significantly reduced (P<0.05). Compared with Model group, the contents of 5-HT, DA and NE in brain tissue of rats in FM group were significantly increased (P<0.05), as shown in Table 10, indicating that the content of neurotransmitters in FM group was increased.

TABLE 10
Effect of the invention on neurotransmitters in rat brain tissue

| Group | 5-HT (ng/ml) | DA (ng/ml) | NE (ng/g) |
|---|---|---|---|
| Control | 8.50 ± 0.37 | 6.56 ± 1.07 | 14.48 ± 1.04 |
| Model | 6.62 ± 0.36* | 4.77 ± 0.52* | 12.65 ± 0.87* |
| Fluoxetine | 7.90 ± 0.54^{#} | 7.06 ± 0.88^{#} | 16.77 ± 1.19^{#} |
| FM | 8.53 ± 0.68^{#} | 7.28 ± 0.58^{#} | 15.86 ± 1.45^{#} |
| ZYDZ1 | 6.76 ± 0.97 | 6.37 ± 0.90^{#} | 13.72 ± 0.90 |
| ZYDZ2 | 7.02 ± 0.73 | 5.46 ± 0.80 | 14.05 ± 1.40 |
| ZYDZ3 | 8.25 ± 0.79^{#} | 5.37 ± 0.60 | 12.94 ± 1.57 |
| ZYDZ4 | 7.31 ± 0.59 | 7.08 ± 0.69^{#} | 14.00 ± 0.78 |
| ZYDZ5 | 8.10 ± 0.70^{#} | 5.70 ± 0.72 | 14.07 ± 2.14 |
| ZYDZ6 | 9.07 ± 0.66^{#} | 6.92 ± 0.91^{#} | 12.86 ± 1.68 |
| ZYDZ7 | 7.18 ± 0.66 | 6.25 ± 1.14^{#} | 13.67 ± 1.21 |
| ZYDZ8 | 6.43 ± 0.40 | 4.37 ± 0.79 | 13.14 ± 0.91 |
| ZYDZ9 | 8.15 ± 0.51^{#} | 5.27 ± 0.64 | 13.84 ± 1.37 |
| ZYDZ10 | 7.28 ± 0.74 | 6.19 ± 0.92^{#} | 15.69 ± 1.30^{#} |
| ZY DZ11 | 8.10 ± 0.59^{#} | 5.53 ± 0.59 | 15.70 ± 0.57^{#} |
| ZY DZ12 | 7.38 ± 0.37 | 6.53 ± 0.65^{#} | 14.48 ± 0.78^{#} |
| ZY DZ13 | 8.77 ± 0.26^{#} | 5.36 ± 1.28 | 14.98 ± 0.74^{#} |
| ZY DZ14 | 6.70 ± 0.38 | 5.69 ± 0.78 | 13.22 ± 1.09 |
| ZY DZ15 | 8.39 ± 0.38^{#} | 5.64 ± 0.84 | 13.86 ± 0.46 |
| ZY DZ16 | 7.19 ± 0.49 | 5.86 ± 0.62 | 15.42 ± 1.05^{#} |
| ZY DZ17 | 7.26 ± 0.57 | 5.77 ± 0.75 | 14.41 ± 0.42^{#} |
| ZY DZ18 | 8.43 ± 0.28^{#} | 4.71 ± 0.67 | 13.68 ± 1.57 |
| ZY DZ19 | 8.44 ± 0.37^{#} | 5.63 ± 0.80 | 14.02 ± 2.06 |

Note:
Compared with Control group, *P < 0.05;
Compared with Model group, ^{#}P < 0.05.

The above research results show that the invention has an obvious regulatory effect on depression related indicators and has a positive effect. Compared with fluoxetine hydrochloride capsule, the effect is similar or even better; Compared with other related Chinese medicine, the invention has better effect. At present, the existing traditional Chinese medicine has a lot of repeated medicine taste, which is generally composed of more than 10 traditional Chinese medicine, or even composed of more than 20 traditional Chinese medicine, which will cause waste of medicine source; The composition of traditional Chinese medicine is complicated and the mechanism is not clear. The invention has less drug taste, reasonable prescription and scientific ratio.

### Clinical Application Case

The purpose of this application case is to discuss the clinical effect of Guanxin Jieyu formula in treating depression syndrome of qi stagnation and blood stasis, heart fire disturbance and uneasiness after coronary heart disease myocardial infarction.

Method: A total of 186 patients with mild to moderate depression after myocardial infarction admitted to the Department of Neurology, the Department of Cardiology, and the Department of Traditional Chinese Medicine outpatient and inpatient departments of the First and Second Affiliated Hospitals of Zhejiang Chinese Medicine University from September 2023 to August 2024 were collected. They were randomly divided into western medicine fluoxetine control group, traditional Chinese medicine Guanxin Jieyu treatment group, and Chinese and Western medicine combined group, with 62 cases in each group. The total course of treatment was 8 weeks. The scores of 24 items Hamilton Depression Scale (HAMD-24), TCM syndrome score, Symptom Checklist 90 (SCL-90) factor standard score and 6-minute walking test distance of the 3 groups were compared before treatment, 4 weeks after treatment and 8 weeks after treatment to analyze the clinical efficacy. Adverse reactions were recorded and safety assessed.

Post myocardial infarction depression (PMID) is characterized by mental retardation, slow thinking, and prolonged mood abnormalities, and is thought to increase the risk of a poor prognosis for myocardial infarction. A number of studies have evaluated the epidemiology of PMID in the past 20 years, but the prevalence reported by different studies varies greatly (13.6%-79.5%). Depression can increase the occurrence of arrhythmia and sudden death in patients with myocardial infarction. Like patients with depression, patients with PMID will also have poor prognosis such as social disorders and even suicidal tendencies due to depression, increasing the medical burden and causing great harm to patients and their families.

### Materials and Methods

General information: From September 2023 to August 2024, 186 patients with PMID syndrome of qi stagnation and blood stasis, heart fire disturbance and mind uneasiness were collected from the Department of Neurology, Cardiology, Outpatient and Inpatient Department of Internal Medicine of Traditional Chinese Medicine, the First and Second Affiliated Hospital of Zhejiang University of Traditional Chinese Medicine. The patients were randomly divided into western medicine fluoxetine control group, Chinese medicine Guanxin Jieyu Decoction treatment group, Chinese and Western medicine combination group. This clinical observation was approved by the Ethics Committee of the Second Affiliated Hospital of Zhejiang University of Traditional Chinese Medicine (Approval No.: 2024 Research No. 046-01).

### Diagnostic Standard

Western diagnostic criteria: Refer to the fourth General Definition of myocardial Infarction published by the European Society of Cardiology (ECS) in 2018. And the Expert Consensus on the Diagnosis and Treatment of Anxiety, Depression and Somatization Symptoms in General Hospitals formulated by the Neuropsychology and Behavioral Neurology Group of the Neurology Branch of the Chinese Medical Association in 2016.

TCM diagnostic criteria: The diagnostic criteria of chest arthralgia and heartache refer to the State Administration of Traditional Chinese Medicine “Criteria for the Diagnosis and Curative Effect of TCM Diseases” and “Guiding Principles for Clinical Research of New TCM Drugs”; The diagnostic criteria of depression syndrome refer to the Criteria for Diagnosis and Curative Effect of TCM Diseases of the State Administration of Traditional Chinese Medicine; Qi stagnation and blood stasis, heart fire upsets, and unrestlessness refer to the National Administration of Traditional Chinese Medicine “Criteria for the Diagnosis and Efficacy of TCM Diseases” and the “13th Five-Year Plan” national textbook for General Higher education “TCM Internal Medicine”.

Inclusion criteria: (1) meet the above diagnostic criteria; (2) did not receive relevant medication one week before treatment; (3) clear consciousness, cooperation in physical examination, no serious cognitive impairment; (4) Hamilton Depression Scale (HAMD-24 item) score ≥8 and ≤35 points, that is, mild to moderate depression patients; (5) Age 25~85 years old, male and female; (6) Patients or family members were informed and signed informed consent, and compliance was good.

Exclusion criteria: Persons with any of the following criteria are not eligible to participate in this study

(1) Patients with a history of clearly diagnosed mental illness or who are taking psychiatric drugs; (2) Patients with severe cardiac insufficiency, liver insufficiency, renal insufficiency, respiratory failure, tumor and severe cerebrovascular disease; (3) Women who are pregnant, breastfeeding or preparing for pregnancy within 3 months; (4) People who are allergic or have been allergic to multiple drugs, or are allergic to the ingredients in the investigational drugs; (5) Patients who participated in other clinical investigators in the 3 months prior to the trial, and who were not considered suitable for participation in the study by other investigators; (6) Incomplete information (such as general information including family history, personal history, past history, drug use history, etc.); (7) Patients who refused to undergo examinations related to this study; (8) Those who do not want to sign the informed consent.

Exclusion and shedding criteria: (1) Patients with serious adverse reactions or other sudden critical illnesses during the study period; (2) Patients who took drugs during the study period that affected the study results; (3) Patients who stopped taking medication without reason and did not cooperate with the study during the study period.

### Method

### Treatment Method

Western medicine control group: Western medicine routine antidepressant treatment. Among the antidepressants, fluoxetine hydrochloride capsule (Paoxetine, Lilly Suzhou Pharmaceutical Co., LTD., National Drug approval number J20181215) was taken orally once a day in the morning, 20 mg/time. The course of treatment was 8 weeks.

Traditional Chinese medicine treatment group: The Guanxin Jieyu Agreement Formula was used for treatment. This formula mainly consists of seven ingredients: 15 g of Salviae miltiorrhizae radix et rhizoma, 9 g of Sappan lignum, 30 g of Ostreae concha, 9 g of Dalbergiae odoriferae lignum, 12 g of Forsythiae fructus, 12 g of raw Ophiopogonis radix, and 0.25 g of Borneolum syntheticum. All these ingredients were uniformly decocted and sealed by the hospital and then provided to the patients. The treatment course lasts for 8 weeks.

Chinese and Western medicine combination group: The traditional Chinese medicine Guanxin Jieyu protocol and Western medicine fluoxetine hydrochloride capsule were given at the same time, the specific method is the same as above. The course of treatment was 8 weeks.

Basic treatment: According to the condition of the subjects, conventional myocardial infarction treatment was given, including anti-platelet aggregation (aspirin, etc.), β-blocker (betalox, etc.), anti-cardiac remodeling ACEI or ARB drugs, plaque stabilization statins and other standard drug therapy.

### Observation Index

(1) The total clinical effective rate and cure rate (cure rate+significant efficiency) of the three groups were compared;
(2) The differences of HAMD-24 scores, TCM syndrome scores, SCL-90 scores, and 6-minute walking test distance among the three groups were compared. The forms were truthfully filled in according to the patients' conditions, and evaluated once before treatment, the fourth week, and the eighth week of treatment;
(3) The number of clinical adverse reactions in the three groups during treatment was observed and compared for safety assessment.

### Criterion of Curative Effect

(1) The evaluation criteria of depression efficacy refer to the Manual of Psychiatric Rating Scale of Modern Psychiatry Series. Hamilton Depression Scale score (HAMD-24) was used to assess the degree of depression. HAMD scores >35 are classified as major depression, 21-35 as moderate depression, 8-20 as mild depression, and <8 as no depression.

The clinical effect was evaluated by the reduction rate: \textcircled{1} Cure: HAMD score <8; Obvious effect: HAMD score reduction rate >50%; Effective: 25%<HAMD score reduction rate ≤50%; Invalid: HAMD score reduction rate ≤25%.
(2) The evaluation criteria of TCM syndrome efficacy refer to the “Guiding Principles for Clinical Research of New Chinese Medicine”, and quantitative observation of TCM syndrome classification was performed on depression, depression, chest tightness and hypochondria, easy breathing or not thinking about food and drink, irritability and crying, foreign body sensation in the throat, less sleep and more dreams, thinking and good doubt, etc., with no score of 0; Occasionally 2 points; Often 4; It's always a 6.
(3) SCL-90 self-rating scale, SCL-90 contains a wide range of psychiatric symptomatology content, testing 9 factors, namely: somatization, obsessive symptoms, interpersonal sensitivity, depression, anxiety, hostility, fear, paranoia and psychosis. Each item evaluated itself on a scale of 1-5, which was as follows: 1: No, felt no problem; 2 points: very light, conscious of the symptoms, but not frequent, life is not affected; 3 points: moderate, conscious of having this symptom, frequent severity from light to moderate; 4 points: severe, conscious of having this symptom, frequent severity from medium to severe; Score 5: Severe, the intensity of all aspects of the symptoms feel very serious.

Factor standard score=(sum of scores of subscale items)/total number of subscale items, the higher the score, the worse the state of the psycho-psychological dimension. The therapeutic effect was evaluated according to the change of SCL-90 factor standard score before and after treatment.
(4) Cardiac function assessment: The six-minute walking distance test (6 MWD) can objectively reflect the cardiac function status and reflect the daily activity ability of patients, which is a simple, effective and safe assessment method. Conduct adequate evaluation before the start of the test to eliminate risk factors that may occur during the test. Accompanied by two physicians, patients were instructed to walk back and forth on flat roads with landmarks for walking distance, and the walking speed was determined by patients according to their own physical ability. The recording staff reported the time every 2 minutes, and recorded the possible discomfort of the patient such as chest tightness and shortness of breath. When the patient was physically exhausted or had discomfort symptoms, the test should be temporarily rested or suspended. The duration of the experiment was 6 minutes, and the walking distance of the patients was recorded and evaluated by the recording staff. According to the test criteria set by the USCarvedilol study, 6-minute walking distances <150 m, 150-450 m, and >450 m were classified as severe, moderate, and mild cardiac dysfunction, respectively.
(5) Record adverse reactions: According to the patient's previous condition, combined with the relevant situation of taking drugs, make a comprehensive judgment of adverse reactions, record its specific manifestations and corresponding treatment measures.

Statistical methods: SPSS26.0 statistical software was used for statistical analysis. The measurement data were expressed by mean±standard deviation (±S) (in line with normal distribution) and median (in line with interquart) (not in line with normal distribution) according to whether the data were in line with normal distribution. The analysis of variance was used for normal distribution, and rank sum test was used for non-normal distribution. Counting data is expressed in % and chi-square test is adopted. The results of repeated measurements of the same specimen at different time points were analyzed by A NOVA. P<0.05 indicated that the difference was statistically significant.

### Results

Comparison of general data: Gender, age, underlying diseases, degree of depression, PCI operation and other general data of the three groups were compared (P>0.05), which was comparable. See Table 11.

TABLE 11
Comparison of general data among the three groups

| | Western | Chinese | Chinese and |
|---|---|---|---|
| | medicine | medicine | Western |
| Item | control group | treatment group | medicine group |
| Number of cases | 62 | 62 | 62 |
| Gender (example) | | | |
| Male | 24 | 26 | 27 |
| Female | 38 | 36 | 35 |
| Age (±s, years) | 68.75 ± 7.07 | 69.18 ± 6.05 | 67.32 ± 7.19 |
| Underlying disease | | | |
| (example) | | | |
| Diabetes | 11 | 14 | 10 |
| Hypertension | 28 | 32 | 25 |
| Smoking history | 42 | 39 | 46 |
| Depression degree | | | |
| (example) | | | |
| Mild | 16 (26%) | 11 (18%) | 16 (26%) |
| Moderate | 46 (74%) | 51 (82%) | 46 (74%) |
| PCI (example) | | | |
| Yes | 42 | 37 | 40 |
| No | 20 | 25 | 22 |

Comparison of HAMD scores: HAMD scores of patients in 3 groups after 4 weeks and 8 weeks of treatment were significantly lower than before treatment, and HAMD scores of patients in 3 groups after 8 weeks of treatment were lower than those in this group after 4 weeks of treatment (P<0.05). After 4 weeks of treatment, compared with the western medicine control group, the HAMD score of the Chinese medicine treatment group and the combined Chinese and Western medicine group were significantly decreased (P<0.05), and the HAMD score of the combined Chinese and Western medicine group was significantly decreased compared with the traditional Chinese medicine treatment group (P<0.05). After 8 weeks of treatment, compared with the western medicine control group, the HAMD score of the Chinese medicine treatment group and the combined Chinese and Western medicine group were significantly decreased (P<0.05), and the HAMD score of the combined Chinese and Western medicine group was not significantly different from that of the traditional Chinese medicine treatment group (P>0.05). The results are shown in Table 12 and FIG. 2.

TABLE 12
Comparison of HAMD scores among three groups (±S)

| | Pre- | Treatment for | Treatment for |
|---|---|---|---|
| Group | treatment | 4 weeks | 8 weeks |
| Western medicine | 22.15 ± 4.79 | 16.08 ± 2.80^{Δ} | 12.15 ± 3.24^{Δ▴} |
| control group | | | |
| Chinese medicine | 23.21 ± 4.50 | 13.74 ± 3.14^{Δ} | 9.11 ± 2.89^{Δ▴} |
| treatment group | | | |
| Chinese and | 23.63 ± 5.14 | 13.21 ± 3.09^{Δ#} | 7.60 ± 2.3^{Δ▴#}* |
| Western medicine | | | |
| group | | | |

Note:
Compared with this group before treatment, ^{Δ}P < 0.05;
Compared with this group after 4 weeks of treatment, ^{▴}P < 0.05;
Compared with western medicine control group, ^{#}P < 0.05;
Compared with the same period Chinese medicine treatment group *P < 0.05.

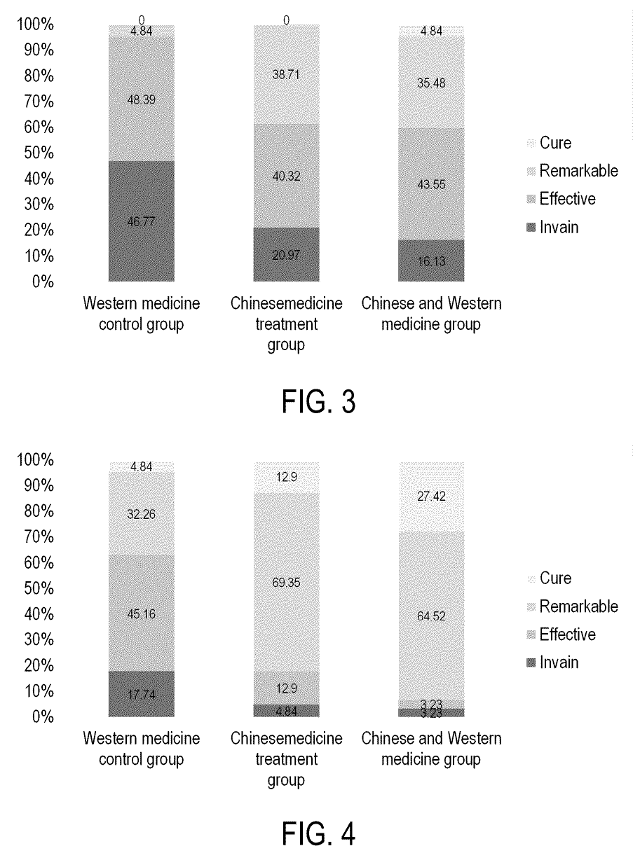

The more apparent rate and total effective rate of HAMD treatment evaluation: The more apparent rate of 3 groups after 8 weeks of treatment was significantly higher than that of this group after 4 weeks of treatment (P<0.05), and the total effective rate had no significant difference (P>0.05). After 4 weeks of treatment, compared with the western medicine control group, the apparent rate of treatment was higher in the combination group (P<0.05), but there was no difference in the Chinese medicine treatment group (P>0.05). There was no significant difference between the treatment group and the TCM group (P>0.05). After 8 weeks of treatment, compared with the western medicine control group, the more apparent rate was higher in the combination group (P<0.05), and the total effective rate had no significant difference (P>0.05), and the more apparent rate and total effective rate in the Chinese medicine treatment group had no significant difference (P>0.05). There were no significant differences in the healing efficiency and total effective rate between the combined treatment group and the TCM treatment group (P>0.05). The results are shown in Table 13, FIG. 3 and FIG. 4.

TABLE 13
Comparison of clinical efficacy among three groups [example (%)]

| | Western | Chinese | Chinese and |
|---|---|---|---|
| | medicine | medicine | Western medicine |
| Group | control group | treatment group | group |
| Treatment | | | |
| time | Treatment for 4 weeks | | |
| Cure | 0 (0.0) | 0 (0.0) | 3 (4.84) |
| Remarkable | 3 (4.84) | 24 (38.71) | 22 (35.48) |
| Effective | 30 (48.39) | 25 (40.32) | 27 (43.55) |
| In vain | 29 (46.77) | 13 (20.97) | 10 (16.13) |
| Cure and | 3 (4.84) | 24 (38.71) | 25 (40.32) ^{#} |
| Remarkable | | | |
| rate | | | |
| Total effective | 33 (53.23) | 49 (79.03) | 52 (83.87) ^{#} |
| rate | | | |
| Treatment | | | |
| time | Treatment for 8 weeks | | |
| Cure | 3 (4.84) | 8 (12.90) | 17 (27.42) |
| Remarkable | 20 (32.26) | 43 (69.35) | 40 (64.52) |
| Effective | 28 (45.16) | 8 (12.90) | 2 (3.23) |
| In vain | 11 (17.74) | 3 (4.84) | 2 (3.23) |
| Cure and | 23 (37.10) ^{▴} | 51 (82.26) ^{▴} | 58 (91.94) ^{▴#} |
| Remarkable | | | |
| rate | | | |
| Total effective | 51 (82.26) | 59 (95.16) | 60 (96.77) _{#} |
| rate | | | |

Note:
Compared with western medicine control group, ^{#}P < 0.05; Compared with this group after 4 weeks of treatment, ^{▴}P < 0.05.

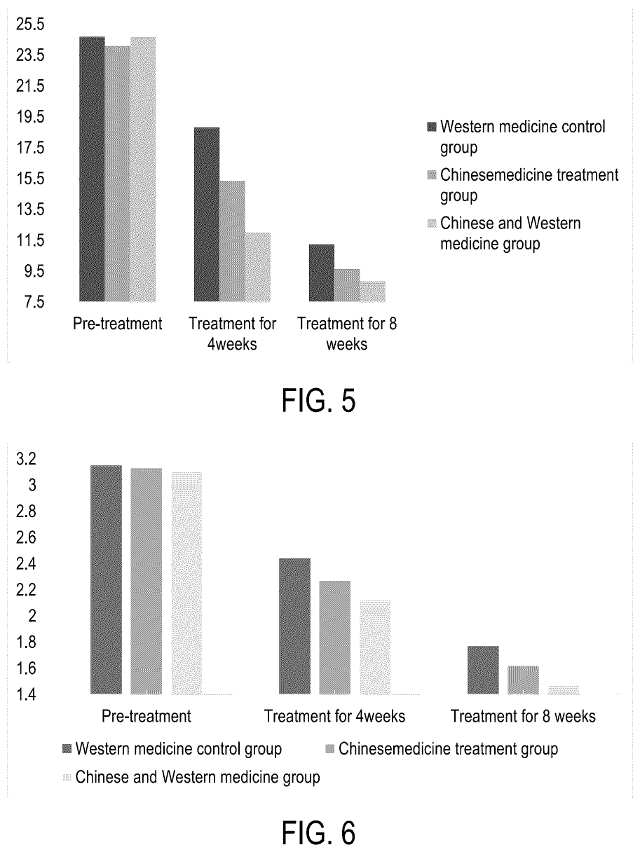

Comparison of TCM syndrome scores: the TCM syndrome scores of 3 groups after 4 weeks and 8 weeks of treatment were significantly decreased compared with that before treatment, and the TCM syndrome scores of 3 groups after 8 weeks of treatment were lower than that of this group after 4 weeks of treatment (P<0.05). After 4 weeks of treatment, compared with the western medicine control group, the TCM syndrome scores in the TCM treatment group and the combined TCM treatment group were significantly decreased (P<0.05), and the TCM syndrome scores in the combined TCM treatment group were significantly decreased compared with the traditional Chinese medicine treatment group (P<0.05). After 8 weeks of treatment, compared with the western medicine control group, TCM syndrome scores in the TCM treatment group and the combined TCM and Western medicine group were significantly decreased (P<0.05), and TCM syndrome scores in the combined TCM and Western medicine group were significantly decreased compared with the traditional Chinese medicine treatment group (P<0.05). The results are shown in Table 14 and FIG. 5.

TABLE 14
Comparison of TCM syndrome scores among three groups (±S)

| | Pre- | Treatment for | Treatment for |
|---|---|---|---|
| Group | treatment | 4 weeks | 8 weeks |
| Western medicine | 24.68 ± 4.78 | 18.81 ± 3.41^{Δ} | 11.23 ± 4.61^{Δ▴} |
| control group | | | |
| Chinese medicine | 24.1 ± 4.12 | 15.35 ± 3.74^{Δ} | 9.65 ± 3.91^{Δ▴} |
| treatment group | | | |
| Chinese and | 24.71 ± 3.53 | 12.03 ± 3.72^{Δ#}* | 8.87 ± 4.42^{Δ▴#} |
| Western medicine | | | |
| group | | | |

Note:
Compared with western medicine control group, ^{Δ}P < 0.05;
Compared with this group after 4 weeks of treatment, ^{▴}P < 0.05;
Compared with western medicine control group, ^{#}P < 0.05;
Compared with the same period Chinese medicine treatment group, *P < 0.05.

Symptom Self-Rating Scale (SCL-90) factor score comparison: the SCL-90 factor score of 3 groups after 4 weeks and 8 weeks of treatment was significantly lower than that before treatment, and the SCL-90 factor score of 3 groups after 8 weeks of treatment was lower than that of this group after 4 weeks of treatment (P<0.05). After 4 weeks of treatment, compared with the western medicine control group, the standard score of SCL-90 factor in the Chinese medicine treatment group and the combined Chinese and Western medicine group was significantly decreased (P<0.05), and the standard score of SCL-90 factor in the combined Chinese and Western medicine group was significantly decreased compared with the traditional Chinese medicine treatment group (P<0.05). After 8 weeks of treatment, compared with western medicine control group, SCL-90 factor standard scores in the combined Chinese and Western medicine group were significantly decreased (P<0.05), but there was no significant difference in the traditional Chinese medicine control group (P<0.05). The standard score of SCL-90 factor in the combined treatment group was not significantly different from that in the Chinese medicine treatment group (P>0.05). The results are shown in Table 15 and FIG. 6.

TABLE 15
Comparison of SCL-90 results among three groups (±S)

| | Pre- | Treatment for | Treatment for |
|---|---|---|---|
| Group | treatment | 4 weeks | 8 weeks |
| Western medicine | 3.15 ± 0.32 | 2.44 ± 0.32^{Δ} | 1.77 ± 0.24^{Δ▴} |
| control group | | | |
| Chinese medicine | 3.13 ± 0.34 | 2.27 ± 0.30^{Δ} | 1.62 ± 0.22^{Δ▴} |
| treatment group | | | |
| Chinese and | 3.11 ± 0.31 | 2.13 ± 0.29^{Δ#}* | 1.48 ± 0.24^{Δ▴#} |
| Western medicine | | | |
| group | | | |

Note:
Compared with this group before treatment, ^{Δ}P < 0.05;
Compared with this group after 4 weeks of treatment, ^{▴}P < 0.05;
Compared with western medicine control group, ^{#}P < 0.05;
Compared with the same period Chinese medicine treatment group *P < 0.05.

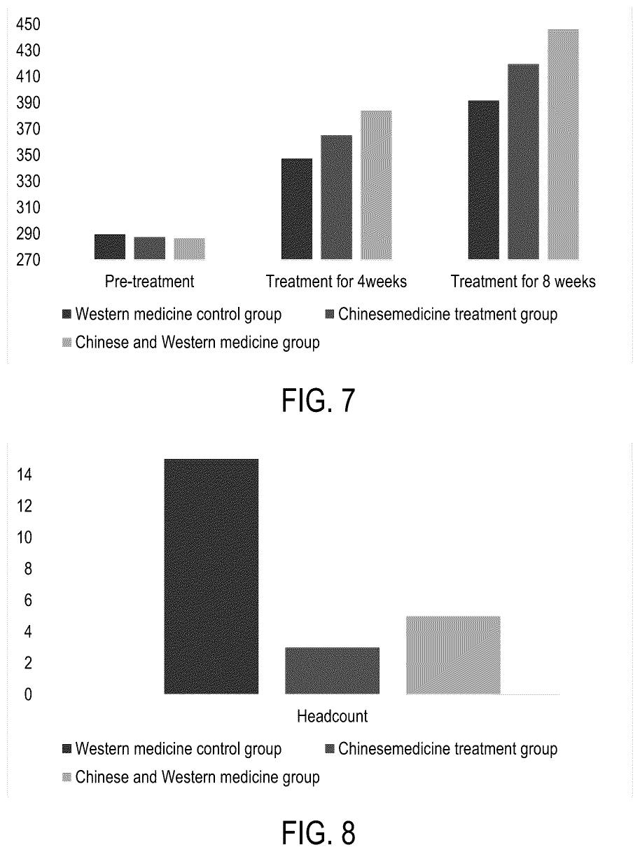

Comparison of 6-minute walking test distance: The 6-minute walking test distance of patients in 3 groups after 4 weeks and 8 weeks of treatment was significantly reduced compared with that before treatment, and the 6-minute walking test distance of patients in 3 groups after 8 weeks of treatment was lower than that of patients in this group after 4 weeks of treatment (P<0.05). After 4 weeks of treatment, compared with the western medicine control group, the six-minute walking test distance of the combined Chinese and Western medicine group was significantly reduced (P<0.05), but there was no significant difference in the Chinese medicine treatment group (P>0.05). There was no significant difference in the 6-minute walking distance between the combined treatment group and the TCM treatment group (P>0.05). After 8 weeks of treatment, compared with the western medicine control group, the six-minute walking test distance of the combined Chinese and Western medicine group was significantly reduced (P<0.05), but there was no significant difference in the Chinese medicine treatment group (P>0.05). There was no significant difference in the 6-minute walking distance between the combined treatment group and the TCM treatment group (P>0.05). The results are shown in Table 16 and FIG. 7.

TABLE 16
Three groups of patients had a six-minute walking distance (±S)

| | Pre- | Treatment for | Treatment for |
|---|---|---|---|
| Group | treatment | 4 weeks | 8 weeks |
| Western medicine | 289.42 ± 74.02 | 347.39 ± 75.10^{Δ} | 391.63 ± 69.53^{Δ▴} |
| control group | | | |
| Chinese medicine | 287.37 ± 72.63 | 365.16 ± 71.49^{Δ} | 419.48 ± 79.93^{Δ▴} |
| treatment group | | | |
| Chinese and | 286.56 ± 78.58 | 384.16 ± 75.58^{Δ#} | 446.32 ± 88.07^{Δ▴#} |
| Western medicine | | | |
| group | | | |

Note:
Compared with this group before treatment, ^{Δ}P < 0.05;
Compared with this group after 4 weeks of treatment, ^{▴}P < 0.05;
Compared with western medicine control group, ^{#}P < 0.05.

Safety assessment: There were no serious adverse reactions in the three groups. During the treatment period, the safety of the TCM treatment group was higher than that of the western medicine control group (P<0.05), and there was no significant difference between the TCM treatment group and the combined group (P>0.05). The safety of combined Chinese and western medicine group was higher than that of western medicine control group, and there was statistical significance (P<0.05). The results are shown in Table 17 and FIG. 8.

TABLE 17
Three groups of security assessment [[example (%)]

| | Dizziness | | Gastro- | | Drowsiness | | | |
|---|---|---|---|---|---|---|---|---|
| | and | | intestinal | | and | | | |
| Group | headache | | reaction | | lethargy | | Total | |
| Western medicine | 7 | (11.3) | 5 | (8.1) | 3 | (8.1) | 15 | (24.2) |
| control group | | | | | | | | |
| Chinese medicine | 0 | (0.0) | 2 | (3.2) | 1 | (1.6) | 3* | (9.7) |
| treatment group | | | | | | | | |
| Chinese and | 1 | (1.6) | 3 | (4.8) | 1 | (1.6) | 5^{#} | (9.7) |
| Western medicine | | | | | | | | |
| group | | | | | | | | |

Note:
Compared with western medicine control group after treatment, ^{#}P < 0.05;
Compared with western medicine control group after treatment, *P < 0.05.

### Discuss

The research results of the invention show that: No matter after 4 weeks or 8 weeks of treatment, HAMD score, TCM symptom score, SCL-90 factor standard score and 6-minute walking test distance of patients in the TCM treatment group and the combined TCM treatment group were significantly improved compared with the western medicine control group. At 4 weeks and 8 weeks of treatment, the total effective rate of the TCM treatment group and the combined TCM treatment group was higher than that of the Western medicine control group. It shows that the clinical therapeutic effect of Chinese medicine alone or combined with Western medicine is stronger than that of Western medicine alone. In the whole treatment process, the number of adverse reactions in the TCM treatment group and the combined TCM and Western medicine group was significantly less than that in the western medicine control group, suggesting that when PMID patients are averse to the adverse reactions caused by Western medicine treatment and do not want to take Western medicine to treat PMID, pure Chinese medicine treatment regimen or the combination of TCM and Western medicine regimen can be used for patients to achieve the purpose of treating diseases and reducing adverse reactions. After 8 weeks of treatment, the HAMD score and SCL-90 factor standard score of the patients were lower than before treatment, and the six-minute walking test distance was longer than before treatment, indicating that Guanxin Jieyu prescription had the effect of restoring the heart function of the patients, alleviating the negative emotions of the patients, and improving the quality of life. In the treatment process of PMID, we should actively explore the treatment mode of traditional Chinese medicine, make full use of the advantages of long-term conditioning and small adverse reactions of traditional Chinese medicine, pay attention to the economic affordability and individual differences of patients, and formulate personalized treatment plans, in order to achieve the best treatment effect and improve the quality of life of patients.

To sum up, Guanxin Jieyu formula has significant efficacy in treating depression after coronary heart disease myocardial infarction with qi stagnation and blood stasis, heart fire disturbance and uneasiness, significantly improving patients' clinical symptoms, relieving patients' mental tension and improving quality of life, with no obvious adverse reactions, safe medication and good patient compliance. It is an effective formula for treating depression after coronary heart disease myocardial infarction, and is worthy of promotion and application.

What is claimed is:

1. A Chinese medicine composition for a treatment of post-coronary heart disease depression, wherein the Chinese medicine composition consists of following components in weight:
1,500-1,600 servings of Ostreae concha, 700-800 servings of Salviae miltiorrhizae radix et rhizoma, 600-700 servings of Forsythiae fructus, 400-500 servings of Dalbergiae odoriferae lignum, 600-700 servings of Ophiopogonis radix, 400-500 servings of Sappan lignum, and 10-11 servings of Borneolum syntheticum.

2. A preparation method of the Chinese medicine composition according to claim 1, wherein the preparation method includes following steps:
step 1) taking a prescribed amount of Forsythiae fructus and Dalbergiae odoriferae lignum, soaking them in water, extracting with steam distillation, collecting a volatile oil, and coating the volatile oil and borneol with β-cyclodextrin to obtain a β-cyclodextrin-coated volatile oil and Borneolum syntheticum for separate use, after centrifuging and removing impurities from an extracted liquid, collecting a residue and extracting respectively for separate use;
step 2) taking an amount of Ostreae concha and adding water to boil, starting with a liquid boiling time, frying for 1 h to obtain a liquid;
step 3) after decocting of the Step 2), adding a formula amount of Salviae miltiorrhizae radix et rhizoma, Sappan lignum, Ophiopogonis radix and the residue of the step 1) to a decocting solution, reflux extraction to obtain reflux extraction solution, centrifuge separation and purification of the reflux extraction solution, combining with an extraction solution of the step 1), concentrating under pressure, and obtaining clear paste;
step 4) adding the volatile oil and Borneolum syntheticum wrapped in β-cyclodextrin prepared in the step 1) into the clear paste, spray dry to obtain dry powder, and then adding auxiliary materials to obtain particles by dry method.

3. The preparation method of the traditional Chinese medicine composition according to claim 2, wherein an amount of water added in the step 1) is eight times a total amount of Forsythiae fructus and Dalbergiae odoriferae lignum, a soaking time is 0.5 hours, and steam distillation is carried out for 7 hours.

4. The preparation method of the traditional Chinese medicine composition according to claim 2, wherein a solid-liquid ratio of Ostreae concha to water in the step 2) is 1 g: 30 L.

5. The preparation method of the Chinese medicine composition according to claim 2, wherein reflux extraction twice in the step 3), each time for 0.5 h, a temperature of decompressive concentration is 50-60° C., and a relative density of the clear paste is 1.10±0.02.

6. A Use of the traditional Chinese medicine compositions according to claim 1 in a preparation of drugs for a treatment of post-coronary depression.
